(19) United States
(12) Patent Application Publication
(10) Pub. No.: US 2010/0114235 A1
(43) Pub. Date: May 6, 2010

(54) HYBRID BATTERY SYSTEM FOR IMPLANTABLE CARDIAC THERAPY DEVICE

(75) Inventors: Naixiong Jiang, Mountain View, CA (US); Gene A. Bornzin, Simi Valley, CA (US); Joseph Beauvais, Liberty, SC (US)

Correspondence Address: STEVEN M MITCHELL;PACESETTER INC, 701 EAST EVELYN AVENUE, SUNNYVALE, CA, 94086, US

(73) Assignee: PACESETTER INC., Sunnyvale, CA (US)

(21) Appl. No.: 12/263,337

(22) Filed: Oct. 31, 2008

(51) Int. Cl. A61N 1/00; H02J 7/00

(52) U.S. Cl. 607/34; 320/103

(57) ABSTRACT

A system and method for powering an implantable cardiac therapy device (ICTD) uses a hybrid battery system. In an embodiment, the hybrid battery system includes of a first type of power cell and a second type of power cell. The first power cell is configured to power low voltage, low current background operations of the ICTD. The second power cell is configured to power high voltage, high current cardiac shocking. The second power cell is further configured to be charged by the first power cell via a continuous, non-regulated charging process, thereby reducing the complexity of the charging circuitry. The system is further configured so that when cardiac shocking is in progress, only the secondary power cell powers the shocking capacitor(s) of the ICTD, and the first power cell is electrically isolated from the shocking capacitor(s). This configuration contributes to longer battery life of the hybrid battery system.

### RELATED APPLICATIONS

This application is related to co-pending and commonly-owned U.S. patent applicaton Ser. No. ______, filed on even date herewith, entitled “Hybrid Battery System With Bioelectric Cell For Implantable Cardiac Therapy Device”, (attorney docket number A07E3046 [1587.1870000]), which is incorporated by reference herein in its entirety as if reproduced in full below.

### BACKGROUND

1. Field of the Invention

The present invention relates generally to implantable cardiac therapy devices, and to power sources for the same. More particularly, the invention relates to a hybrid battery system for use in an implantable cardiac therapy device.

2. Background Art

Implantable cardiac therapy devices (ICTDs) enjoy widespread use for providing convenient, portable, sustained therapy for cardiac patients with a variety of cardiac arrhythmias. ICTDs may combine a pacemaker and defibrillator in a single implantable device. Such devices may be configured to provide ongoing cardiac pacing in order to maintain an appropriate cardiac rhythm. In addition, should the ICTD detect that the patient is experiencing an episode of ventricular fibrillation (or an episode of ventricular tachycardia), the ICTD can deliver appropriate defibrillation therapy.

An ICTD requires a portable power supply in the form of a battery. The battery has several inherent requirements including safety and also the ability to provide power to the ICTD for an extended period of time, thereby minimizing the frequency of invasive procedures to replace the battery.

However, ICTDs have additional, specialized power requirements due to the specific nature of their function. Long-term cardiac pacing can be supported by a low voltage, low current power source. Defibrillation therapy, however, requires rapid, high voltage, high current delivery to the heart. There does not exist a single battery which is optimized to effectively provide both types of electrical sourcing.

Presently, the lithium/silver vanadium oxide battery (LiSVO battery) is a common power source for ICTDs. The LiSVO battery is capable of producing high power pulses and charging the capacitors of the device in a timely manner. Further, the LiSVO battery has a high energy density (which, in theory, provides long battery life), and its self-discharge rate is low.

However, the LiSVO battery suffers from disadvantages as well. Its internal resistances from both the anode and cathode tend to increase as the battery discharges over time, particularly during midlife. As a result, over time, the loaded voltage will be lower and the time for charging the shocking capacitors will be longer. In some cases, the time to charge the shocking capacitors could be doubled, which may render the battery unacceptable for defibrillation. This may result in a medical decision to replace the device, which in turn means the patient may have to accept a premature surgery. In the past, the increased battery charge time has been a major issue for ICTDs.

A recent improvement has been the use of a hybrid battery source. A hybrid battery system combines two different physical batteries, with different but complementary electrical properties, into a single functional package. The single functional package effectively serves as the battery for the ICTD. A first physical battery (which may also be referred to as a cell) of the hybrid battery typically has a high energy density for long battery life, but may have a relatively low voltage and/or current output. A second physical battery (or cell) has higher peak current delivery capability (typically a result of lower internal resistance), and may have a higher voltage output, and superior recharging time and recharging properties. However, the second cell typically has lower energy density that the first battery. The two cells are coupled in the hybrid battery, with the first cell providing charging to the second cell.

A hybrid battery with the indicated architecture has been described, for example, by Greatbatch (see U.S. Pat. No. 7,079,893 B2, issued Jul. 18, 2006). However, existing hybrid batteries may still not be optimally tuned for application in an ICTD. For example, the voltage output or output current of the second cell may not be as high as desirable. The second cell may also have undesirable properties associated with recharging (for example, it may not be safe to charge the second cell too quickly), requiring complex regulation circuitry. (Section 6 of this document, “System and Method For Hybrid Battery Optimized for ICTD,” provides a discussion and characterization of a “regulated charging process” and an “unregulated charging process.”)

In addition, full advantage may not be taken of the electrical properties of the primary cell. Furthermore, existing hybrid batteries may not have an optimized energy density distribution (that is, an optimized distribution of storage capacity) between the primary and secondary cells. Finally, the secondary cell may introduce an undesirable degree of bulk or weight in the design of the ICTD.

What is needed, then, is a hybrid battery design which is optimized in terms of electrical properties, structural properties, and operational properties, for use in an implantable cardiac therapy device.

### BRIEF SUMMARY

The present system and method employs a hybrid battery comprised of at least two types of cells to power an implantable cardiac therapy device (ICTD). A first type of cell provides low voltage but high energy density. The first type of cell directly provides power to the ICTD for purposes of routine cardiac monitoring, pacing, and general low current ICTD operations (including, for example, communications). The first type of cell is also coupled to a second cell via a simple DC-to-DC converter. The second type of cell is maintained at full or nearly full charge by the energy provided by the first type of cell. The second type of cell has low internal resistance and high voltage, making it suitable to rapidly charge ICTD capacitors for cardiac shocking (that is, for defibrillation). The second type of cell also has other properties optimizing it for usage in an ICTD.

An optimized energy density distribution may be implemented between the first type of cell and the second type of cell. In one embodiment, the first type of cell is a LiMnO₂ battery, while the second type of cell is a Li ion polymer battery. Each type of cell may be implemented as a single physical cell, or alternatively as two or more physical cells of the same type.

Further embodiments, features, and advantages of the present system and method, as well as the structure and operation of the various embodiments of the present system and method, are described in detail below with reference to the accompanying drawings.

### BRIEF DESCRIPTION OF THE DRAWINGS/FIGURES

The accompanying drawings, which are incorporated herein and form part of the specification, illustrate the methods and systems presented herein for a hybrid battery optimized for an implantable cardiac therapy device. Together with the detailed description, the drawings further serve to explain the principles of and to enable a person skilled in the relevant art(s) to make and use the methods and systems presented herein.

In the drawings, like reference numbers indicate identical or functionally similar elements. Further, the drawing in which an element first appears is typically indicated by the leftmost digit(s) in the corresponding reference number (e.g., an element numbered 302 first appears in FIG. 3).

Additionally, some elements may be labeled with only a number to indicate a generic form of the element, while other elements labeled with the same number followed by another number or a letter (or a letter/number combination) may indicate a species of the element. A period or underscore may be introduced in the label for clarity of reading, and has no other significance.

FIG. 1 is a simplified diagram illustrating an exemplary implantable cardiac therapy device (ICTD) in electrical communication with a patient's heart by means of leads suitable for delivering multi-chamber stimulation and pacing therapy, and for detecting cardiac electrical activity.

FIG. 2 is a functional block diagram of an exemplary ICTD that can detect cardiac electrical activity and analyze cardiac electrical activity, as well as provide cardioversion, defibrillation, and pacing stimulation in four chambers of a heart.

FIG. 3 is a functional block diagram of the internal architecture and principle external connections of an exemplary external programming device which may be used by a human programmer to monitor or program an ICTD.

FIG. 4 is functional block diagram of an exemplary hybrid battery system, along with interconnections to some elements of an exemplary ICTD, according to an embodiment of the present system and method.

FIG. 5 is functional block diagram of an exemplary hybrid battery system, along with interconnections to some elements of an exemplary ICTD, according to an embodiment of the present system and method.

FIG. 6 is an exploded view of an exemplary hybrid battery system according to an embodiment of the present system and method.

FIG. 7 shows a set of experimentally measured plots of the time required for various Li ion polymer cells to charge shocking capacitors in a representative ICTD.

FIG. 8 shows a set of experimentally measured plots of the time required for various Li ion polymer cells to charge shocking capacitors in a representative ICTD.

FIG. 9 shows a set of experimentally measured plots of the time required for a representative Li ion polymer cell to charge the shocking capacitors of a representative ICTD at different current levels.

### DETAILED DESCRIPTION

1. Overview
2. Exemplary Environment—Overview
3. Exemplary ICTD in Electrical Communication with a Patient's Heart
4. Functional Elements of an Exemplary ICTD
5. ICTD Programmer
6. System and Method For Hybrid Battery Optimized for ICTD
7. Choice of Power Cells
8. Lithium Ion Polymer Cell vs. Lithium/Silver Vanadium Oxide Cell
9. Lithium Ion Polymer Cell vs. Standard Lithium Ion Cell
10. Charging of Lithium Ion Polymer Cell from Primary Cell
11. Relative Storage Capacities of Different Types of Cells
12. Alternative Embodiments
13. Conclusion

### 1. Overview

The following detailed description of systems and methods for a hybrid battery optimized for an implantable cardiac therapy device refers to the accompanying drawings that illustrate exemplary embodiments consistent with these systems and methods. Other embodiments are possible, and modifications may be made to the embodiments within the spirit and scope of the methods and systems presented herein. Therefore, the following detailed description is not meant to limit the methods and systems described herein. Rather, the scope of these methods and systems is defined by the appended claims.

It would be apparent to one of skill in the art that the systems and methods for a hybrid battery optimized for an implantable cardiac therapy device, as described below, may be implemented in many different embodiments of hardware, software, firmware, and/or the entities illustrated in the figures. Any actual hardware and/or software described herein is not limiting of these methods and systems. In addition, more than one embodiment of the present system and method may be presented below, and it will be understood that not all embodiments necessarily exhibit all elements, that some elements may be combined or connected in a manner different than that specifically described herein, and that some differing elements from the different embodiments presented herein may be functionally and structurally combined to achieve still further embodiments of the present system and method.

Thus, the operation and behavior of the methods and systems will be described with the understanding that modifications and variations of the embodiments are possible, given the level of detail presented herein.

### 2. Exemplary Environment—Overview

Before describing in detail the methods and systems for a hybrid battery optimized for an implantable cardiac therapy device, it is helpful to describe an example environment in which these methods and systems may be implemented. The methods and systems described herein may be particularly useful in the environment of an implantable cardiac therapy device (ICTD).

An ICTD may also be referred to synonymously herein as a “stimulation device”, emphasizing the role of the ICTD in providing pacing and shocking to a human heart. However, an ICTD may provide operations or services in addition to stimulation, including but not limited to cardiac monitoring.

An ICTD is a physiologic measuring device and therapeutic device that is implanted in a patient to monitor cardiac function and to deliver appropriate electrical therapy, for example, pacing pulses, cardioverting and defibrillator pulses, and drug therapy, as required. ICTDs include, for example and without limitation, pacemakers, cardioverters, defibrillators, implantable cardioverter defibrillators, implantable cardiac rhythm management devices, and the like. Such devices may also be used in particular to monitor cardiac electrical activity and to analyze cardiac electrical activity. The term “implantable cardiac therapy device” or simply “ICTD” is used herein to refer to any such implantable cardiac therapy device.

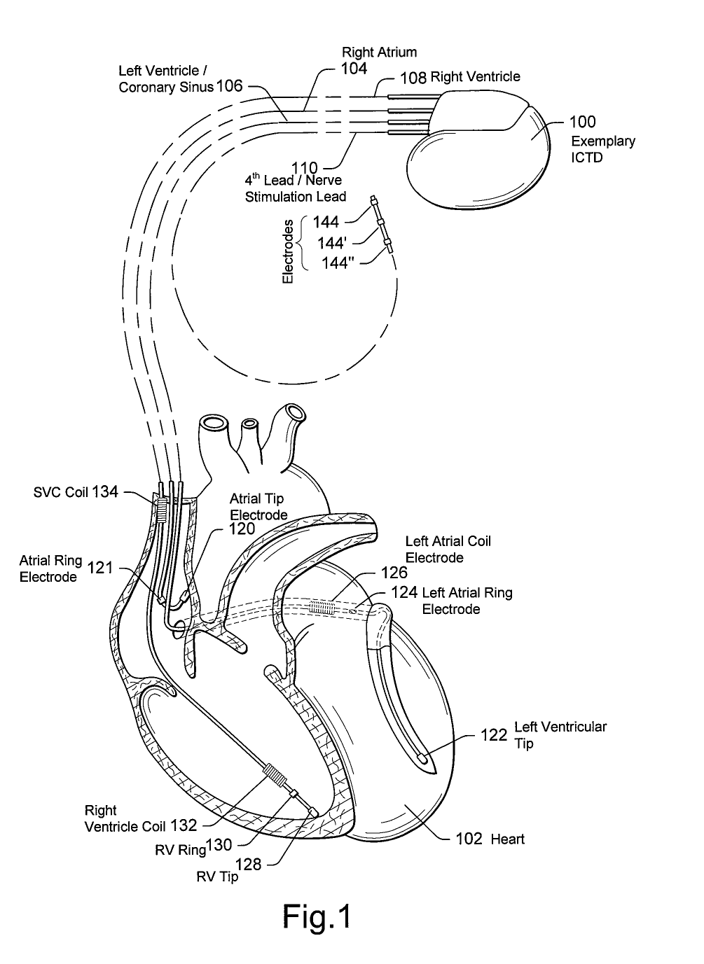

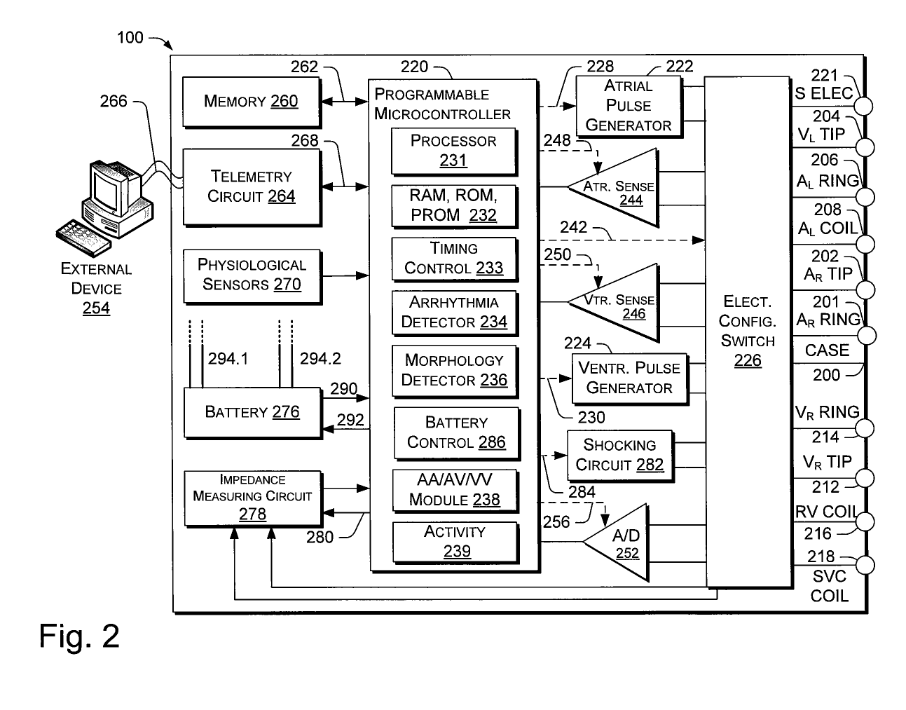

FIGS. 1 and 2 illustrate such an environment.

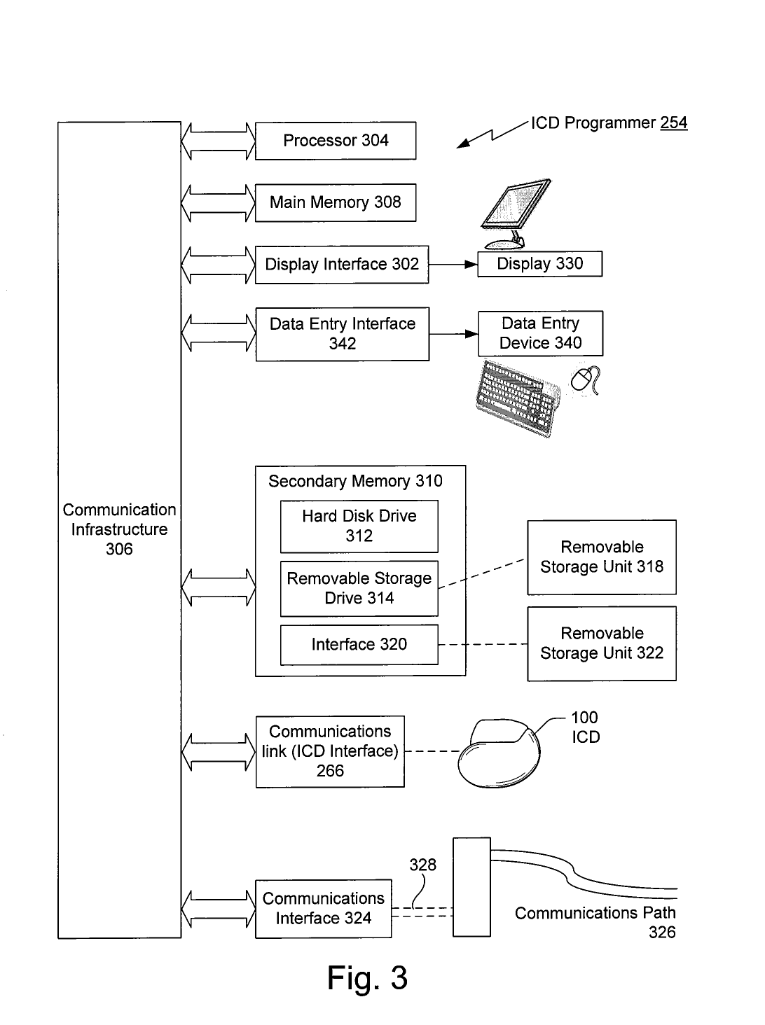

FIG. 3 illustrates the architecture of an external programming device which may be used to monitor, program, or interact with an ICTD.

### 3. Exemplary ICTD in Electrical Communication with a Patient's Heart

The techniques described below are intended to be implemented in connection with any ICTD or any similar stimulation device that is configured or configurable to stimulate nerves and/or stimulate and/or shock a patient's heart.

FIG. 1 shows an exemplary stimulation device 100 in electrical communication with a patient's heart 102 by way of three leads 104, 106, 108, suitable for delivering multi-chamber stimulation and shock therapy. The leads 104, 106, 108 are optionally configurable for delivery of stimulation pulses suitable for stimulation of autonomic nerves. In addition, the device 100 includes a fourth lead 110 having, in this implementation, three electrodes 144, 144′, 144″ suitable for stimulation of autonomic nerves. This lead may be positioned in and/or near a patient's heart or near an autonomic nerve within a patient's body and remote from the heart. Of course, such a lead may be positioned epicardially or at some other location to stimulate other tissue.

The right atrial lead 104, as the name implies, is positioned in and/or passes through a patient's right atrium. The right atrial lead 104 optionally senses atrial cardiac signals and/or provide right atrial chamber stimulation therapy. As shown in FIG. 1, the stimulation device 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for stimulation of autonomic nerves.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

Accordingly, an exemplary coronary sinus lead 106 is optionally designed to receive atrial and ventricular cardiac signals and to deliver left ventricular pacing therapy using, for example, at least a left ventricular tip electrode 122, left atrial pacing therapy using at least a left atrial ring electrode 124, and shocking therapy using at least a left atrial coil electrode 126. For a complete description of a coronary sinus lead, the reader is directed to U.S. Pat. No. 5,466,254, “Coronary Sinus Lead with Atrial Sensing Capability” (Helland), which is incorporated herein by reference. The coronary sinus lead 106 further optionally includes electrodes for stimulation of autonomic nerves. Such a lead may include pacing and autonomic nerve stimulation functionality and may further include bifurcations or legs. For example, an exemplary coronary sinus lead includes pacing electrodes capable of delivering pacing pulses to a patient's left ventricle and at least one electrode capable of stimulating an autonomic nerve. An exemplary coronary sinus lead (or left ventricular lead or left atrial lead) may also include at least one electrode capable of stimulating an autonomic nerve, such an electrode may be positioned on the lead or a bifurcation or leg of the lead.

Stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108 is capable of sensing or receiving cardiac signals, and delivering stimulation in the form of pacing and shock therapy to the right ventricle. An exemplary right ventricular lead may also include at least one electrode capable of stimulating an autonomic nerve, such an electrode may be positioned on the lead or a bifurcation or leg of the lead.

### 4. Functional Elements of an Exemplary ICTD

An implantable cardiac therapy device may be referred to variously, and equivalently, throughout this document as an “implantable cardiac therapy device”, an “ICTD”, an “implantable device”, a “stimulation device”, and the respective plurals thereof.

FIG. 2 shows an exemplary, simplified block diagram depicting various components of stimulation device 100. The stimulation device 100 can be capable of treating both fast and slow arrhythmias with stimulation therapy, including cardioversion, defibrillation, and pacing stimulation. The stimulation device can be solely or further capable of delivering stimuli to autonomic nerves. While a particular multi-chamber device is shown, it is to be appreciated and understood that this is done for illustration purposes only. For example, various methods may be implemented on a pacing device suited for single ventricular stimulation and not bi-ventricular stimulation. Thus, the techniques and methods described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart with cardioversion, defibrillation, pacing stimulation, and/or autonomic nerve stimulation.

Housing 200 for stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 (see FIG. 1) for shocking purposes. Housing 200 further includes a connector (not shown) having a plurality of terminals 201, 202, 204, 206, 208, 212, 214, 216, 218, 221 (shown schematically and, for convenience, the names of the electrodes to which they are connected are shown next to the terminals).

To achieve right atrial sensing, pacing and/or autonomic stimulation, the connector includes at least a right atrial tip terminal (AR TIP) 202 adapted for connection to the atrial tip electrode 120. A right atrial ring terminal (AR RING) 201 is also shown, which is adapted for connection to the atrial ring electrode 121. To achieve left chamber sensing, pacing, shocking, and/or autonomic stimulation, the connector includes at least a left ventricular tip terminal (VL TIP) 204, a left atrial ring terminal (AL RING) 206, and a left atrial shocking terminal (AL COIL) 208, which are adapted for connection to the left ventricular tip electrode 122, the left atrial ring electrode 124, and the left atrial coil electrode 126, respectively. Connection to suitable autonomic nerve stimulation electrodes is also possible via these and/or other terminals (e.g., via a nerve stimulation terminal S ELEC 221).

To support right chamber sensing, pacing, shocking, and/or autonomic nerve stimulation, the connector further includes a right ventricular tip terminal (VR TIP) 212, a right ventricular ring terminal (VR RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively. Connection to suitable autonomic nerve stimulation electrodes is also possible via these and/or other terminals (e.g., via the nerve stimulation terminal S ELEC 221).

At the core of the stimulation device 100 is a programmable microcontroller 220 that controls the various modes of stimulation therapy. As is well known in the art, microcontroller 220 typically includes a processor or microprocessor 231, or equivalent control circuitry, designed specifically for controlling the delivery of stimulation therapy, and may further include onboard memory 232 (which may be, for example and without limitation, RAM, ROM, PROM, one or more internal registers, etc.), logic and timing circuitry, state machine circuitry, and I/O circuitry.

Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander) and U.S. Pat. No. 4,944,298 (Sholder), all of which are incorporated by reference herein. For a more detailed description of the various timing intervals used within the stimulation device and their inter-relationship, see U.S. Pat. No. 4,788,980 (Mann et al.), also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to autonomic nerves or other tissue) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 233 to control the timing of the stimulation pulses (e.g., pacing rate, atrio-ventricular (e.g., AV) delay, atrial interconduction (AA) delay, or ventricular interconduction (VV) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234, a morphology detector 236, and optionally an orthostatic compensator and a minute ventilation (MV) response module (the latter two are not shown in FIG. 2). These components can be utilized by the stimulation device 100 for determining desirable times to administer various therapies, including those to reduce the effects of orthostatic hypotension. The aforementioned components may be implemented in hardware as part of the microcontroller 220, or as software/firmware instructions programmed into the device and executed on the microcontroller 220 during certain modes of operation.

Microcontroller 220 further includes an AA delay, AV delay and/or VV delay module 238 for performing a variety of tasks related to AA delay, AV delay and/or VV delay. This component can be utilized by the stimulation device 100 for determining desirable times to administer various therapies, including, but not limited to, ventricular stimulation therapy, bi-ventricular stimulation therapy, resynchronization therapy, atrial stimulation therapy, etc. The AA/AV/VV module 238 may be implemented in hardware as part of the microcontroller 220, or as software/firmware instructions programmed into the device and executed on the microcontroller 220 during certain modes of operation. Of course, such a module may be limited to one or more of the particular functions of AA delay, AV delay and/or W delay. Such a module may include other capabilities related to other functions that may be germane to the delays. Such a module may help make determinations as to fusion.

The microcontroller 220 of FIG. 2 also includes an activity module 239. This module may include control logic for one or more activity related features. For example, the module 239 may include an algorithm for determining patient activity level, calling for an activity test, calling for a change in one or more pacing parameters, etc. These algorithms are described in more detail with respect to the figures. The module 239 may be implemented in hardware as part of the microcontroller 220, or as software/firmware instructions programmed into the device and executed on the microcontroller 220 during certain modes of operation. The module 239 may act cooperatively with the AA/AV/VV module 238.

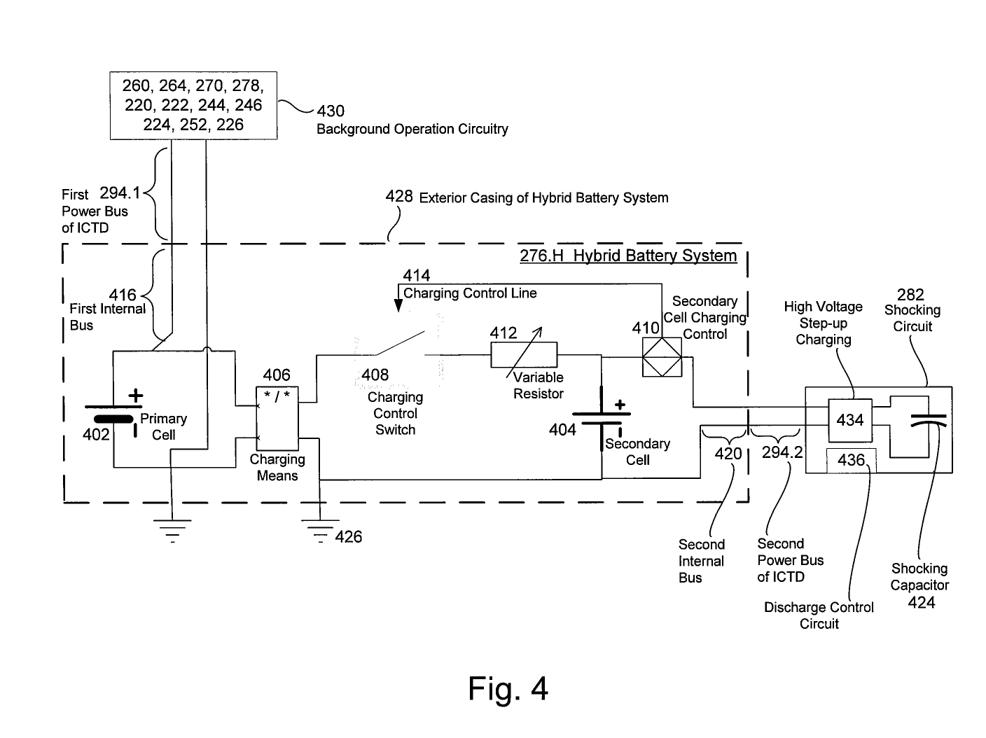

Microcontroller 220 may also include a battery control module 286. Battery control module 286 may be used, for example, to control a battery 276 (which may be a hybrid battery 276.H, illustrated in FIGS. 4, 5, and 6) as discussed in further detail below in this document. Battery control 286 may be hardwired circuitry, or may be implemented as software or firmware running on microcontroller 220. Battery control 286 may be coupled to battery 276 via battery signal line 290 and battery control line 292. Battery signal line 290 may deliver to battery control 286 status or operational information regarding battery 276. Battery control line 292 may be used to change an operational state of battery 276. For example, battery control line 292 may deliver control signals from battery control 286 to battery 276. For example, in an embodiment where battery 276 is a hybrid battery, battery control 286 may send control signals to determine if a second cell is connected to a first cell for recharging of the second cell. The details of this are further discussed below.

In an alternative embodiment, battery control 286 may be a separate module from microcontroller 220, but may be coupled to microcontroller 220. For example, separate module battery control 286 may obtain required ICTD operational status information from microcontroller 220. Or, for example, separate module battery control 286 may report battery status or battery operational information to microcontroller 220. In addition, separate module battery control 286 may also be coupled to battery 276.

In an alternative embodiment, battery control 286 may be implemented as an internal physical module of battery 276 (for example, battery control 286 may be implemented as a microchip which is situated internally to the exterior housing of battery 276). However, battery control 286 may still be coupled to microcontroller 220 via battery signal line 290 and battery control line 292. In an alternative embodiment, battery control functions of battery control 286 may be distributed across a first module which is part of battery 276, and one or more additional modules which are external to battery 276. The battery control module(s) external to battery 276 may for example be part of microcontroller 220.

Battery 276 is discussed in more detail below in this document.

The electrode configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, combipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial (ATR. SENSE) and ventricular (VTR. SENSE) sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the analog-to-digital (A/D) data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, the device 100 utilizes the atrial and ventricular sensing circuits, 244 and 246, to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. In some instances, detection or detecting includes sensing and in some instances sensing of a particular signal alone is sufficient for detection (e.g., presence/absence, etc.).

The timing intervals between sensed events (e.g., P-waves, R-waves, and depolarization signals associated with fibrillation which are sometimes referred to as “F-waves” or “Fib-waves”) are then classified by the arrhythmia detector 234 of the microcontroller 220 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to inputs of an analog-to-digital (A/D) data acquisition system 252. The data acquisition system 252 is configured to acquire intracardiac electrogram (EGM) signals, convert the raw analog data into a digital signal, and store the digital signals for later processing and/or telemetric transmission to an external device 254. Data acquisition system 252 may be configured by microcontroller 220 via control signals 256. The data acquisition system 252 is coupled to the right atrial lead 104, the coronary sinus lead 106, the right ventricular lead 108 and/or the nerve stimulation lead 110 through the switch 226 to sample cardiac signals across any pair of desired electrodes.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature may be the ability to sense and store a relatively large amount of data (e.g., from the data acquisition system 252), which data may then be used for subsequent analysis to guide the programming of the device.

Essentially, the operation of the ICTD control circuitry, including but not limited to pulse generators, timing control circuitry, delay modules, the activity module, battery utilization and related voltage and current control, and sensing and detection circuits, may be controlled, partly controlled, or fine-tuned by a variety of parameters, such as those indicated above which may be stored and modified, and may be set via an external ICTD programming device.

Advantageously, the operating parameters of the implantable device 100 may be non-invasively programmed into the memory 260 through a telemetry circuit 264 in telemetric communication via communication link 266 with the external device 254, such as a general purpose computer, a dedicated ICTD programmer, a transtelephonic transceiver, or a diagnostic system analyzer. The microcontroller 220 activates the telemetry circuit 264 with a control signal 268. The telemetry circuit 264 advantageously allows intracardiac electrograms and status information relating to the operation of the device 100 (as contained in the microcontroller 220 or memory 260) to be sent to the external device 254 through an established communication link 266. The ICTD 100 may also receive human programmer instructions via the external device 254.

The stimulation device 100 can further include a physiologic sensor 270, commonly referred to as a “rate-responsive” sensor because it is typically used to adjust pacing stimulation rate according to the exercise state of the patient. However, the physiological sensor 270 may further be used to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Accordingly, the microcontroller 220 may respond by adjusting the various pacing parameters (such as rate, AA delay, AV delay, VV delay, etc.) at which the atrial and ventricular pulse generators, 222 and 224, generate stimulation pulses.

While shown as being included within the stimulation device 100, it is to be understood that the physiologic sensor 270 may also be external to the stimulation device 100, yet still be implanted within or carried by the patient. Examples of physiologic sensors that may be implemented in device 100 include known sensors that, for example, sense respiration rate, pH of blood, ventricular gradient, cardiac output, preload, afterload, contractility, hemodynamics, pressure, and so forth. Another sensor that may be used is one that detects activity variance, wherein an activity sensor is monitored diurnally to detect the low variance in the measurement corresponding to the sleep state. For a complete description of an example activity variance sensor, the reader is directed to U.S. Pat. No. 5,476,483 (Bornzin et al.), issued Dec. 19, 1995, which patent is hereby incorporated by reference.

More specifically, the physiological sensors 270 optionally include sensors for detecting movement and minute ventilation in the patient. The physiological sensors 270 may include a position sensor and/or a minute ventilation (MV) sensor to sense minute ventilation, which is defined as the total volume of air that moves in and out of a patient's lungs in a minute. Signals generated by the position sensor and MV sensor are passed to the microcontroller 220 for analysis in determining whether to adjust the pacing rate, etc. The microcontroller 220 monitors the signals for indications of the patient's position and activity status, such as whether the patient is climbing upstairs or descending downstairs or whether the patient is sifting up after lying down.

The stimulation device additionally includes a battery 276 that provides operating power to all of the circuits shown in FIG. 2, as well as to any additional circuits which may be present in alternative embodiments. Operating power in the form of electrical current and/or voltage may be provided via a power bus or power buses 294, depicted in FIG. 2 as a first power bus 294.1 and a second power bus 294.2. In FIG. 2, the connection(s) of power bus(es) 294 to other elements of ICTD 100 for purposes of powering those elements is not illustrated, but is implied by the dotted end-lines of bus(es) 294.

For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 Amps, at voltages above 2 volts, for periods of 10 seconds or more). In an embodiment, discussed in detail later in this document, battery 276 may be configured to provide a current as high as 3.5 to 4.5 Amps and/or unloaded voltages in excess of 4 volts, for rapid charging of shocking circuitry. Battery 276 also desirably has a predictable discharge characteristic so that elective replacement time can be determined.

In an embodiment, battery 276 may be a hybrid battery comprised of dual types of cells, as described further below. Such a hybrid battery may provide power via a plurality of power buses, such as buses 249.1 and 294.2 of FIG. 2. In an embodiment, each power bus may be configured to deliver different voltages, different currents, and/or different power levels. Battery 276 may be monitored and/or controlled via battery control 286, as discussed in part above, and as also discussed further below.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuit 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 so that any desired electrode may be used.

In the case where the stimulation device 100 is intended to operate as an implantable cardioverter/defibrillator (ICTD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to approximately 0.5 J), moderate (e.g., approximately 0.5 J to approximately 10 J), or high energy (e.g., approximately 11 J to approximately 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV coil electrode 132, or as part of a split electrical vector using the SVC coil electrode 134 or the left atrial coil electrode 126 (i.e., using the RV electrode as a common electrode). Other exemplary devices may include one or more other coil electrodes or suitable shock electrodes (e.g., a LV coil, etc.).

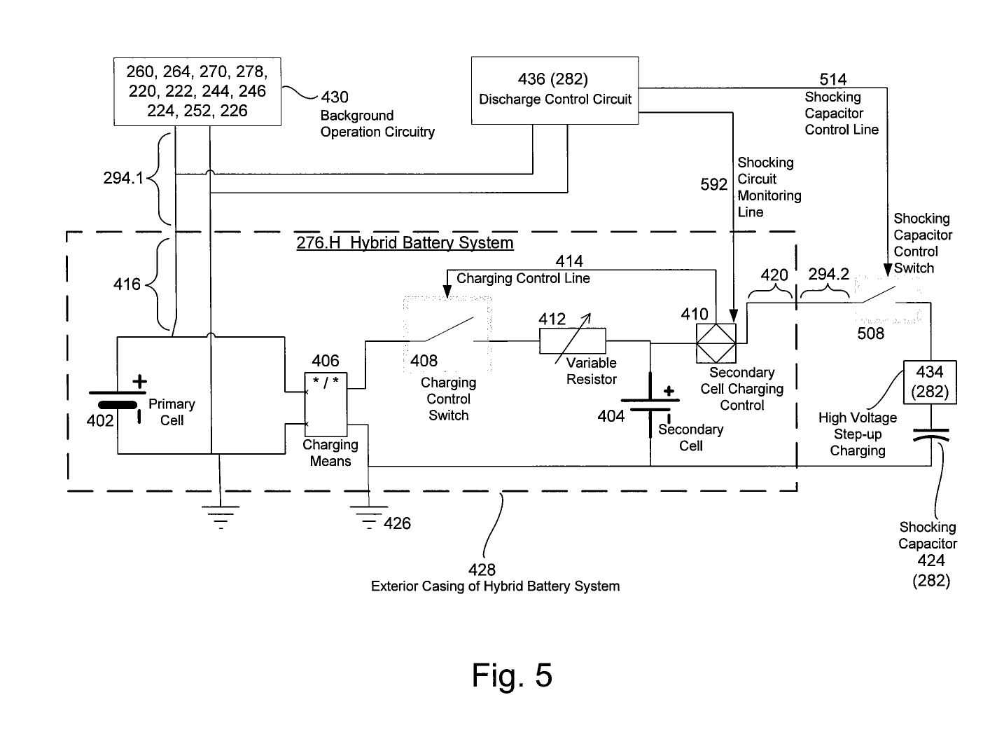

Shocking circuit 282 either has within it, or is coupled to, one or more shocking capacitors (not shown in FIG. 2, but see for example element 424 of FIGS. 4 and 5). The shocking capacitor(s) 424 may be used to store up energy, and then release that energy, during the generation of shocking pulses.

Cardioversion level shocks are generally considered to be of low to moderate energy level (where possible, so as to minimize pain felt by the patient), and/or synchronized with an R-wave and/or pertaining to the treatment of tachycardia. Defibrillation shocks are generally of moderate to high energy level (i.e., corresponding to thresholds in the range of approximately 5 J to approximately 40 J), delivered asynchronously (since R-waves may be too disorganized), and pertaining exclusively to the treatment of fibrillation. Accordingly, microcontroller 220 is capable of controlling the synchronous or asynchronous delivery of the shocking pulses.

### 5. ICTD Programmer

As indicated above, the operating parameters of the implantable device 100 may be non-invasively programmed into the memory 260 through a telemetry circuit 264 in telemetric communication via communication link 266 with the external device 254. The external device 254 may be a general purpose computer running custom software for programming the ICTD 100, a dedicated external programmer device of ICTD 100, a transtelephonic transceiver, or a diagnostic system analyzer. Generically, all such devices may be understood as embodying computers, computational devices, or computational systems with supporting hardware or software which enable interaction with, data reception from, and programming of ICTD 100.

Throughout this document, where a person is intended to program or monitor ICTD 100 (where such person is typically a physician or other medical professional or clinician), the person is always referred to as a “human programmer” or as a “user”. The term “human programmer” may be viewed as synonymous with “a person who is a user of an ICTD programming device”, or simply with a “user”. Any other reference to “programmer” or similar terms, such as “ICTD programmer”, “external programmer”, “programming device”, etc., refers specifically to the hardware, firmware, software, and/or physical communications links used to interface with and program ICTD 100.

The terms “computer program”, “computer code”, and “computer control logic” are generally used synonymously and interchangeably in this document to refer to the instructions or code which control the behavior of a computational system. The term “software” may be employed as well, it being understood however that the associated code may in some embodiments be implemented via firmware or hardware, rather than as software in the strict sense of the term (e.g., as computer code stored on a removable medium, or transferred via a network connection, etc.).

A “computer program product” or “computational system program product” is a medium (for example, a magnetic disk drive, magnetic tape, optical disk (e.g., CD, DVD), firmware, ROM, PROM, flash memory, a network connection to a server from which software may be downloaded, etc) which is suitable for use in a computer or computation system, or suitable for input into a computer or computational system, where the medium has control logic stored therein for causing a processor of the computational system to execute computer code or a computer program. Such medium, also referred to as “computer program medium”, “computer usable medium”, and “computational system usable medium”, are discussed further below.

FIG. 3 presents a system diagram representing an exemplary computer, computational system, or other programming device, which will be referred to for convenience as ICTD programmer 254. It will be understood that while the device is referred to an “ICTD programmer”, indicating that the device may send programming data, programming instructions, programming code, and/or programming parameters to ICTD 100, the ICTD programmer 254 may receive data from ICTD 100 as well, and may display the received data in a variety of formats, analyze the received data, store the received data in a variety of formats, transmit the received data to other computer systems or technologies, and perform other tasks related to operational and/or physiologic data received from ICTD 100.

ICTD programmer 254 includes one or more processors, such as processor 304. Processor 304 is used for standard computational tasks well known in the art, such as retrieving instructions from a memory, processing the instructions, receiving data from memory, performing calculations and analyses on the data in accordance with the previously indicated instructions, storing the results of calculations back to memory, programming other internal devices within ICTD programmer 254, and transmitting data to and receiving data from various external devices such as ICTD 100.

Processor 304 is connected to a communication infrastructure 306 which is typically an internal communications bus of ICTD programmer 254; however, if ICTD programmer 254 is implemented in whole or in part as a distributed system, communication infrastructure 306 may further include or may be a network connection.

ICTD programmer 254 may include a display interface 302 that forwards graphics, text, and other data from the communication infrastructure 306 (or from a frame buffer not shown) for display on a display unit 330. The display unit may be, for example, a CRT, an LCD, or some other display device. Display unit 330 may also be more generally understood as any device which may convey data to a human programmer.

Display unit 330 may also be used to present a user interface which displays internal features of, operating modes or parameters of, or data from ICTD 100. The user interface presented via display unit 330 of ICTD programmer 254 may include various options that may be selected, deselected, or otherwise changed or modified by a human programmer of ICTD 100. The options for programming the ICTD 100 may be presented to the human programmer via the user interface in the form of buttons, check boxes, menu options, dialog boxes, text entry fields, or other icons or means of visual display well known in the art.

ICTD programmer 254 may include a data entry interface 342 that accepts data entry from a human programmer via data entry devices 340. Such data entry devices 340 may include, for example and without limitation, a keyboard, a mouse, a touchpad, a touch-sensitive screen, a microphone for voice input, or other means of data entry, which the human programmer uses in conjunction with display unit 330 in a manner well known in the art. For example, either a mouse or keystrokes entered on a keyboard may be used to select check boxes, option buttons, menu items, or other display elements indicating human programmer choices for programming ICTD 100. Direct text entry may be employed as well. Data entry device 340 may also take other forms, such as a dedicated control panel with specialized buttons and/or other mechanical elements or tactile sensitive elements for programming ICTD 100.

In the context of the present system and method, display interface 302 may present on display unit 330 a variety of data related to patient cardiac function and performance, and also data related to the current operating mode, operational state, or operating parameters of ICTD 100. Modifications to ICTD 100 operational state(s) may be accepted via data entry interface 342 and data entry device 340. In general, any interface means which enables a human programmer to interact with and program ICTD 100 may be employed. In one embodiment, for example, a visual data display may be combined with tactile data entry via a touch-screen display.

In another embodiment, a system of auditory output (such as a speaker or headset and suitable output port for same, not shown) may be employed to output data relayed from ICTD 100, and a system of verbal input (such as a microphone and suitable microphone port, not shown) may be employed to program ICTD 100. Other modes of input and output means may be employed as well including, for example and without limitation, a remote interaction with ICTD 100, viewing printed data which has been downloaded from ICTD 100, or the programming of ICTD 100 via a previously coded program script.

All such means of receiving data from ICTD 100 and/or programming ICTD 100 constitute an interface 302, 330, 342, 340 between ICTD 100 and a human programmer of ICTD 100, where the interface is enabled via both the input/output hardware (e.g., display screen, mouse, keyboard, touchscreen, speakers, microphone, input/output ports, etc.) and the hardware, firmware, and/or software of ICTD programmer 254.

ICTD programmer 254 also includes a main memory 308, preferably random access memory (RAM), and may also include a secondary memory 310. The secondary memory 310 may include, for example, a hard disk drive 312 and/or a removable storage drive 314, representing a floppy disk drive, a magnetic tape drive, an optical disk drive, etc. The removable storage drive 314 reads from and/or writes to a removable storage unit 318 in a well known manner. Removable storage unit 318 represents a magnetic disk, magnetic tape, optical disk, etc. which is read by and written to by removable storage drive 314. As will be appreciated, the removable storage unit 318 includes a computer usable storage medium having stored therein computer software and/or data.

In alternative embodiments, secondary memory 310 may include other similar devices for allowing computer programs or other instructions to be loaded into ICTD programmer 254. Such devices may include, for example, a removable storage unit 322 and an interface 320. Examples of such may include a program cartridge and cartridge interface (such as that found in video game devices), a removable memory chip (such as an erasable programmable read only memory (EPROM), programmable read only memory (PROM), or flash memory) and associated socket, and other removable storage units 322 and interfaces 320, which allow software and data to be transferred from the removable storage unit 322 to ICTD programmer 254.

ICTD programmer 254 also contains a communications link 266 to ICTD 100, which may be comprised in part of a dedicated port of ICTD programmer 254. From the perspective of ICTD programmer 254, communications link 266 may also be viewed as an ICTD interface. Communications link 266 enables two-way communications of data between ICTD programmer 254 and ICTD 100. Communications link 266 has been discussed above (see the discussion of FIG. 2).

ICTD programmer 254 may also include a communications interface 324. Communications interface 324 allows software and data to be transferred between ICTD programmer 254 and other external devices (apart from ICTD 100). Examples of communications interface 324 may include a modem, a network interface (such as an Ethernet card), a communications port, a Personal Computer Memory Card International Association (PCMCIA) slot and card, etc. Software and data transferred via communications interface 324 are in the form of signals 328 which may be electronic, electromagnetic, optical or other signals capable of being received by communications interface 324. These signals 328 are provided to communications interface 324 via a communications path (e.g., channel) 326. This channel 326 carries signals 328 and may be implemented using wire or cable, fiber optics, a telephone line, a cellular link, an radio frequency (RF) link and other communications channels.

The terms “computer program medium”, “computer usable medium”, and “computational system usable medium” are used, synonymously, to generally refer to media such as removable storage drive 314, a hard disk installed in hard disk drive 312, and removable storage units 318 and 322. These computer program products or computational system program products provide software to ICTD programmer 254.

It should be noted, however, that it is not necessarily the case that the necessary software, computer code, or computer program (any of which may also referred to as computer control logic) be loaded into ICTD programmer 254 via a removable storage medium. Such computer program may be loaded into ICTD programmer 254 via communications link 328, or may be stored in memory 308 of ICTD programmer 254. Computer programs are stored in main memory 308 and/or secondary memory 310. Computer programs may also be received via communications interface 324.

Accordingly, such computer programs represent controllers of ICTD programmer 254, and thereby controllers of ICTD 100. Software may be stored in a computer program product and loaded into ICTD programmer 254 using removable storage drive 314, interface 320, hard drive 312 or communications interface 324.

In an embodiment of the present system and method, ICTD programmer 254 may be used to modify ICTD operating parameters of battery control 286. In this way, ICTD programmer 254 may be used to modify the operations of a battery 276, such as a hybrid battery discussed in further detail below.

### 6. System and Method For Hybrid Battery Optimized for ICTD

FIG. 4 is a schematic diagram of an exemplary hybrid battery system 276.H according to the present system and method. FIG. 4 also includes some elements of exemplary connections between exemplary hybrid battery system 276.H and other elements of ICTD 100.

In an embodiment, an exemplary hybrid battery system 276.H may be comprised of an exemplary primary cell 402 and an exemplary secondary cell 404. In an alternative embodiment, two or more primary cells 402 may be employed. In an alternative embodiment, two or more secondary cells 404 may be employed.

In an embodiment, primary cell 402 may be a lithium-magnesium oxide (Li/MnO₂) cell. In an alternative embodiment, primary cell 402 may be a lithium carbon monofluoride (LiCFₓ) cell. In an embodiment, secondary cell 404 may be a lithium ion polymer cell. The present system and method may enjoy several advantages due to the specific selections of power cells. These advantages are discussed in detail below in this document in the section entitled “Choice of Power Cells”.

Primary cell 402 and secondary cell 404 may be coupled by charging means 406. Further coupled between charging means 406 and secondary cell 404 may be a charging control switch 408 and a variable resistor 412. In an embodiment, and as shown in FIG. 4, primary cell 402 and secondary cell 404 may be coupled in parallel. Secondary cell 404 may also be coupled to a secondary cell charging control circuit 410, which may also be known as a charging control circuit 410. Secondary cell charging control circuit 410 may be, for example, a programmable logic control (PLC) circuit.

Secondary cell charging control circuit 410 may further be coupled to shocking circuit 282 of ICTD 100. Secondary cell charging control circuit 410 may also be coupled to charging control switch 408 via charging control line 414.

In an embodiment, secondary cell charging control circuit 410 is internal to hybrid battery system 276.H, and therefore contained within exterior casing 428. In an alternative embodiment (not illustrated in FIG. 4), secondary cell charging control circuit 410 may be external to hybrid battery system 276.H, and may for example comprise or be part of battery control module 286 of ICTD 100 (discussed above in conjunction with FIG. 2). In the latter embodiment, secondary cell charging control circuit 410 may be coupled to hybrid battery system 276.H, and in particular to charging control switch 408, via suitable control lines such as battery control line 292 (see FIG. 2). By way of exemplary embodiments, the discussion below assumes that secondary cell charging control circuit 410 is internal to hybrid battery system 276.H unless otherwise indicated.

An additional element of hybrid battery system 276.H may be a first internal bus 416 which is coupled to primary cell 402. First internal bus 416 is configured to be coupled to first power bus 294.1 of ICTD 100. In turn, first power bus 294.1 may be connected to numerous elements of ICTD 100 already discussed above. These elements may include, for example and without limitation, memory 260, telemetry circuit 264, physiologic sensor 270, impedance measuring circuit 278, microcontroller 220, atrial pulse generator 222, atrial sensing circuits 244, ventricular sensing circuits 246, analog-to-digital converter 252, and electrode configuration switch 226. Collectively, these elements and similar elements of ICTD 100 may be referred to as background operation circuitry 430.

It is an advantage of the present system and method that background operation circuitry 430 is powered via the lower voltage primary cell 402 rather than the higher voltage secondary cell 404. Background operations, such as cardiac pacing and monitoring, can typically be powered at lower currents and voltages than cardiac shocking (for example, at approximately 2 volts for pacing, as opposed to approximately 4 volts for shocking). If low voltage background circuits 430 are run off a high voltage cell (for example, if background activities are run off a high voltage secondary cell 404), then energy is wasted, reducing overall battery life. In the alternative, the voltage from a high powered cell could be stepped down to run lower voltage background operations, but power would be lost here as well. Running low voltage background circuits 430 off the low voltage primary cell 402 ensures overall longer life of hybrid battery system 276.H.

Hybrid battery system 276.H may also include a second internal bus 420 which is coupled to secondary cell 404. Second internal bus 420 may be configured to be coupled to a second power bus 294.2 of ICTD 100. Second power bus 294.2 may be coupled to shocking circuit 282 of ICTD 100. Shocking circuit 282 may include, among other elements, one or more shocking capacitor(s) 424. Shocking capacitor(s) 424 may be charged via the power provided from secondary cell 404 via second internal bus 420 and second power bus 294.2.

Shocking circuit 282 typically also includes means of high voltage step-up charging 434, such as a flyback charging circuit 434. Charging circuit 434 accepts current from secondary cell 404, and charges shocking capacitor(s) 424 to high voltages (typically in excess of 800 volts). This ensures that a high current necessary for cardiac shocking can be supplied from shocking capacitor(s) 424.

In an embodiment, shocking circuit 282 may include a discharge control circuit 436, which is configured to control and/or regulate the discharge of shocking capacitor(s) 424 for cardiac shocking. Discharge control circuit 436 may in turn be programmed by, controlled partly or wholly by, or work in conjunction with control signals from microcontroller 220 of ICTD 100. Discharge control circuit 436 may for control purposes be coupled to high voltage step-up charging 434, to shocking capacitors 424, and/or to microcontroller 220 in ways which will be apparent to persons skilled in the relevant arts (coupling not illustrated in FIG. 4). In an alternative embodiment, the functions of discharge control circuit 436 may be provided entirely by microcontroller 220 of ICTD 100, with microcontroller 220 being suitably coupled to high voltage step-up charging 434 and/or shocking capacitor 424.

Additional elements of hybrid battery system 276.H may include connections or leads to grounding elements 426. Grounding elements 426 may, for example, be the exterior housing or “can” 200 of ICTD 100.

In operational use when hybrid battery system 276.H is installed in an operational ICTD 100, primary cell 402 may provide continuous low voltage, low current power to background operation circuitry 430 of ICTD 100.

Primary cell 402, as already noted, may be coupled to background operation circuitry 430 via first internal bus 416 of battery 276.H and first power bus 294.1 of ICTD 100.

Primary cell 402 may also be coupled via charging means 406 to secondary cell 404. When secondary cell 404 is not in use for shocking a patient, then in typical operation charging control switch 408 is closed. With charging control switch 408 closed, primary cell 402 and secondary cell 404 are coupled in parallel. Further, with charging control switch 408 closed, primary cell 402 and secondary cell 404 are configured so that secondary cell 404 may be continuously charged via charging means 406.

It is an advantage of the present system and method that because secondary cell 404 is a lithium ion polymer cell, it may be possible to continually charge secondary cell 404. Other possible types of secondary cells, such as, for example, a standard lithium ion cell (which is not a lithium ion polymer cell), may require careful regulation of the charging process. For example, regulation may be required to ensure that the other types of secondary cells do not charge too rapidly, or do not overcharge. Charging too rapidly or overcharging may damage these other types of secondary cells and may even result in rupture or burning of the secondary cell.

Regulation of the charging process may take the form of monitoring the charge on the secondary cell 404, and stopping the charging process when secondary cell 404 is fully charged. Continued monitoring may be required to determine when secondary cell 404 has lost charge (for example, due to self-discharge over time), requiring that the charging process be started again. Alternatively, the rate of charging, for example, the rate of current flow from primary cell 402 to secondary cell 404, may need frequent adjustment to prevent overcharging of secondary cell 404. Additional circuitry and cost may be entailed to provide for such monitoring and regulation of the charging process.

However, in an embodiment of the present system and method, when a secondary cell 404 is a lithium ion polymer cell, it may be possible to charge secondary cell 404 from primary cell 402 at a steady, continuous rate. Put another way, primary cell 402 may transfer power to secondary cell 404 via a continuous charging. As a result, there may be no requirement for complex regulation circuitry to turn the charging process on or off, or to reduce the rate of the charging process. Secondary cell 404 may be continuously charged from primary cell 402, or put another way, secondary cell 404 may be charged from primary cell 402 via an unregulated charging process.

As used herein, an “unregulated charging process” is a charging process where there is no requirement for circuitry or for a method to monitor and adjust the charging process on account of the possibility of overcharge of secondary cell 404. The term “unregulated charging process” is not intended to refer to the operation of charging means 406 but only to the regulation of the charging of secondary cell 404 to prevent an overcharging condition. For example, a person skilled in the art will recognize that charging means 406 may be implemented as a regulated DC-to-DC converter which will use feedback to regulate the output voltage at a desired voltage level. Such regulation is separate and apart from overcharge regulation.

Even with an unregulated charging process, it may be desirable to establish a rate of current flow, for example, to set a maximum limit to the current from primary cell 402 to secondary cell 404. This limit may be set, for example, via variable resistor 412. The phrase “unregulated charging process” may be further understood to mean that such a maximum limit to the current flow, once set, does not need to be further regulated or controlled over time in order to prevent overcharge or damage to secondary cell 404. The maximum permitted current flow from primary cell 402 to secondary cell 404 may be set, for example, as part of a fixed design element of hybrid battery system 276.H. Or, for example, the maximum permitted current flow from primary cell 402 to secondary cell 404 may be set on a per unit basis (that is, per individual specimen of hybrid battery system 276.H) during an initial configuration or set up of hybrid battery system 276.H.

Charging means 406 may be, for example, a DC-to-DC converter 406. In an embodiment of the present system and method, DC-to-DC converter 406 may be a precision converter, meaning that the converter is configured to deliver a specific voltage level to a high degree of precision. No other charging circuitry may be required to charge secondary cell 404 from primary cell 402. In an embodiment of the present system and method, primary cell 402 may put out a voltage on the order of two volts. DC-to-DC converter 406 steps up this voltage to a voltage above four volts, such as to a voltage of 4.1 volts or 4.2 volts. In this way secondary cell 404 is maintained at a voltage, such as, for example, approximately 4.1 to 4.2 volts, which is substantially above the voltage of primary cell 402. In an embodiment of the present system and method, DC-to-DC converter 406 is configured for high-efficiency voltage conversion, resulting in minimal energy loss. DC-to-DC converters are well known in the art. For example, the DC-to-DC converter may be a capacitive or inductive, switch-mode power converter. Selection and implementation of an appropriate DC-to-DC converter would be apparent to a person skilled in the relevant arts.

Because secondary cell 404 may have a self-discharge process, secondary cell 404 may never reach exactly the voltage level put out by DC-to-DC converter 406. Therefore, even when secondary cell 404 is substantially fully charged, a small charging current may continue to flow from DC-to-DC converter 406 to secondary cell 404. This small current, which may be on the order of 100 μAmps, may be referred to as a “trickle charge”. In some embodiments of the present system and method, battery life of secondary cell 404 may be preserved by preventing the trickle charge. Therefore, in some embodiments of the present system and method, a regulated charging process may be employed. In some embodiments, hybrid battery system 276.H may have charging regulation circuitry (not shown in FIGS. 4 or 5). The charging regulation circuitry may be configured to stop the charging process when the charging current falls below a certain threshold value. The charging regulation circuitry may further be configured to restart the charging process when the voltage on secondary cell 404 falls below a designated voltage level, for example, 4 volts. The charging regulation circuitry may stop or start the charging process by any of several means, such as for example by opening or closing charging control switch 408.

In an embodiment of the present system and method, the choice of whether to configure hybrid battery system 276.H for an unregulated charging process or a regulated charging process may depend on the particular choice of secondary cell 404. For example, the choice may depend on a particular brand of secondary cell 404 which may be employed.

Variable resistor 412 may also be coupled between charging means 416 and secondary cell 404. As discussed above, secondary cell 404 may be charged from primary cell 402 via an unregulated charging process without risk of damage to secondary cell 404, and without risk of harm to the patient in whom ICTD 100 is implanted. However, there may still be a maximum safe current from primary cell 402 to secondary cell 404. Further, primary cell(s) 402 may only be able to safely discharge current provided the current flow from primary cell(s) 402 is below a certain rate, for example, typically on the order of a few milliamperes. Variable resistor 412 may therefore serve the purpose of limiting a rate at which current is drawn from primary cell 402 when primary cell 402 is charging secondary cell 404. The exact resistance of variable resistor 412 may be slowly varied over an extended period of time (such as over periods of several months) via control circuitry (not illustrated) in response to the fact that primary cell 402 slowly loses power over the extended period of time.

In an embodiment of the present system and method, exemplary charging control switch 408 is coupled to secondary cell charging control circuit 410 via exemplary charging control line 414. Charging control switch 408 may, for example, be a transistor such as a field effect transistor (FET), or other switching element well known in the art. In normal operation, when secondary cell 404 is not charging shocking capacitor 424 for shocking purposes, secondary cell charging control circuit 410 maintains charging control switch 408 in a closed state. This enables primary cell 402 to be coupled to secondary cell 404, allowing secondary cell 404 to be charged as already described above.

When secondary cell charging control circuit 410 determines that a shocking process is occurring or is about to occur, secondary cell charging control circuit 410 sends a signal via charging control line 414 to charging control switch 408. The signal causes charging control switch 408 to enter an open state. When charging control switch 408 is open, secondary cell 404 is no longer charged by primary cell 402. Further, with charging control switch 408 open, primary cell 402 is no longer coupled even indirectly to shocking circuit 282 or shocking capacitor 404 of shocking circuit 282.

As a result, during a shocking process, all energy for the shocking process is provided by secondary cell 404, which is optimized to provide power for the shocking process. With charging control switch 408 open, primary cell 402 is electrically isolated from shocking capacitor 404. Therefore, none of the power to shocking capacitor 404 is provided by primary cell 402, which conserves the energy storage of primary cell 402. In this way, the power of primary cell 402 is preserved for those applications for which primary cell 402 is optimized, thereby extending the overall life of hybrid battery system 276.H.

In an embodiment, secondary cell charging control circuit 410 detects that a shocking process is in progress by detecting a power discharge from secondary cell 404 or by detecting a load from shocking circuit 282 via second internal bus 420 and second power bus 294.2.

In an alternative embodiment of the present system and method, not illustrated in the figure, secondary cell charging control circuit 410 may be coupled to discharge control circuit 436 or to microcontroller 220 of ICTD 100 (for example, to battery control element 286 of microcontroller 220). Discharge control circuit 436 or microcontroller 220 (or, specifically, battery control element 286) may send a signal to secondary cell charging control circuit 410 indicating that a shocking process in is progress or is about to commence.

A shocking process may be a single shock or a series of shocks closely spaced in time. For example, a series of shocks may be spaced 5 to 10 seconds apart, though shorter or longer intervals are possible. The exact shocking process, including voltage(s) employed, the number of shocks, and timing of the shocks, may be determined by discharge control circuit 436 of shocking circuit 282, or by microcontroller 220, or by a combination of discharge control circuit 436 and microcontroller 220. Secondary cell charging control circuit 410 may determine that a shocking process has concluded, for example by monitoring the discharge activity of secondary cell 404 and/or by monitoring a power drain of shocking circuit 282 and/or shocking capacitor 424. In an alternative embodiment, secondary cell charging control circuit 410 may determine that a shocking process has concluded by receiving an appropriate signal from discharge control circuit 436 or from microcontroller 220 (for example, from battery control element 286 of microcontroller 220).

When secondary cell charging control circuit 410 has determined that the shocking process is concluded, secondary cell charging control circuit 410 may send a signal via charging control line 414 to charging control switch 408. The signal closes charging control switch 408. This recouples primary cell 402 with secondary cell 404, so that secondary cell 404 may be recharged for future shocking.

FIG. 5 represents an exemplary hybrid battery system 276.H and elements of an associated exemplary ICTD 100 according to another embodiment of the present system and method. Many elements shown in FIG. 5 are the same as elements shown in FIG. 4 and a detailed discussion of them will not be repeated here.

Note that In FIG. 5, and strictly due to considerations of clarity of illustration, elements of shocking circuit 282 are not all shown in immediate proximity to each other as in FIG. 4. Those elements which may be considered part of shocking circuit 282 still include shocking capacitor 424, high voltage step-up charging 434, and discharge control circuit 436. All three elements 424, 434, 436 are also labelledlabeled parenthetically as “(282)” to indicate their inclusion with shocking circuit 282. Persons skilled in the relevant arts will appreciate that the layouts shown in both FIGS. 4 and 5 are schematic in nature, and the inclusion of elements 424, 434, and 436 as part of shocking circuit 282 is dependent on the functional roles, interconnections, and/or interactions of these elements, as opposed to any particular schematic layout selected for purposes of clarity of illustration.

In FIG. 5, ICTD 100 has been configured so that discharge control circuit 436 is now powered via primary cell 402. Discharge control circuit 436 is coupled to primary cell 402 via first internal bus 416 and first power bus 294.1. In general, the elements of shocking circuit 282 which may be powered via primary cell 402 may include elements which pertain to regulation, control, monitoring, activation, termination, and/or timing of the shocking process.

Shocking capacitor 424 is still powered via secondary cell 404. Shocking capacitor 424 may still be coupled to secondary cell 404 via second internal bus 420 and second power bus 294.2. In FIG. 5, the charging of shocking capacitor 424 is controlled by discharge control circuit 436 of shocking circuit 282 (discussed above in more detail in conjunction with FIG. 2 and FIG. 4).

For example, charging of shocking capacitor 424 may be controlled by discharge control circuit 436 via an exemplary shocking capacitor control line 514 which may open or close a shocking capacitor control switch 508. Shocking capacitor control switch 508 may determine whether secondary cell 404 is coupled to shocking capacitor 424. In this way, even though discharge control circuit 436 is powered by primary cell 402, the high voltage and high current shocking capacitor 424 continues to be charged as necessary via secondary cell 404.

Note that in FIG. 5, charging of shocking capacitor 424 from secondary cell 404 is still via high voltage step-up charging means 434. In FIG. 5, high voltage step-up charging 434 is illustrated as being configured between shocking capacitor control switch 508 and shocking capacitor 424. In an alternative embodiment, shocking capacitor control switch 508 may be configured between high voltage step-up charging 434 and shocking capacitor 424, so that shocking capacitor control switch 508 controls the flow of charging current from step-up charging circuit 434 to shocking capacitor 424. Persons skilled in the relevant arts will appreciate that in such an embodiment, suitable changes would be made in the circuit connections to ensure that secondary cell 404 is coupled to charging circuit 434.

Charging control circuit 410 of hybrid battery 276.H still controls whether primary cell 402 is coupled to secondary cell 404. This control is via charging control line 414 and charging control switch 408 as before. Secondary cell charging control circuit 410 may determine if shocking capacitor 424 is being charged by monitoring the activity of shocking capacitor control switch 508 via second internal bus 420 and second power bus 294.2, or via some other control line or monitoring line (not illustrated in FIG. 5).

In an alternative embodiment, secondary cell charging control circuit 410 monitors the shocking activity of discharge control circuit 436 and shocking capacitor 424 via a shocking circuit monitoring line 592 which couples discharge control circuit 436 to secondary cell charging control circuit 410. In an embodiment, shocking circuit monitoring line 592 may be an element of or may be the same as battery control line 292 (discussed above in conjunction with FIG. 2). In an alternative embodiment, shocking circuit monitoring line 592 may be an additional control line apart from battery control line 292.

In summary, when ICTD 100 starts a high current pulse discharge (or series of discharges) for cardiac shocking, secondary cell 404 is disconnected from charging means 406 by secondary cell charging control circuit 410. After the high current pulse discharge or series of charges is over, secondary cell charging control circuit 410 automatically switches secondary cell 404 to be recoupled with primary cell 402, so that secondary cell 404 is charged again by primary cell 402. As discussed further below, the output voltage of charging means 406 is set at approximately 4.1 to 4.2 volts, so that secondary cell 404 can be maintained at a voltage level higher than 4.0 volts.

Persons skilled in the relevant arts will appreciate that while FIGS. 4 and 5 illustrate a single shocking capacitor 424, in embodiments of the present system and method two or more shocking capacitors 424 may be charged via secondary cell 404. As noted above, the charging of shocking capacitors 424 from secondary cell 404 is done via a high voltage step-up converter 434, in order to charge capacitors 424 to hundreds of volts from the approximately 4 volts of secondary cell 404. Implementation of high voltage step-up converter 434 will be apparent to a person skilled in the art and may be, for example, a flyback (buck boost) converter or other topology converter or current source. Additional shocking capacitor control switches 508 and other elements (e.g., control lines, additional power buses, etc.) may be included to support additional capacitors.

Persons skilled in the relevant arts will further appreciate that the exact configurations, connections, and arrangements of electrical components shown in FIG. 4 and FIG. 5 are exemplary only. Additional components, fewer components, alternative components, and variations in the connections may be employed consistent with the system and method for a hybrid battery system described herein.

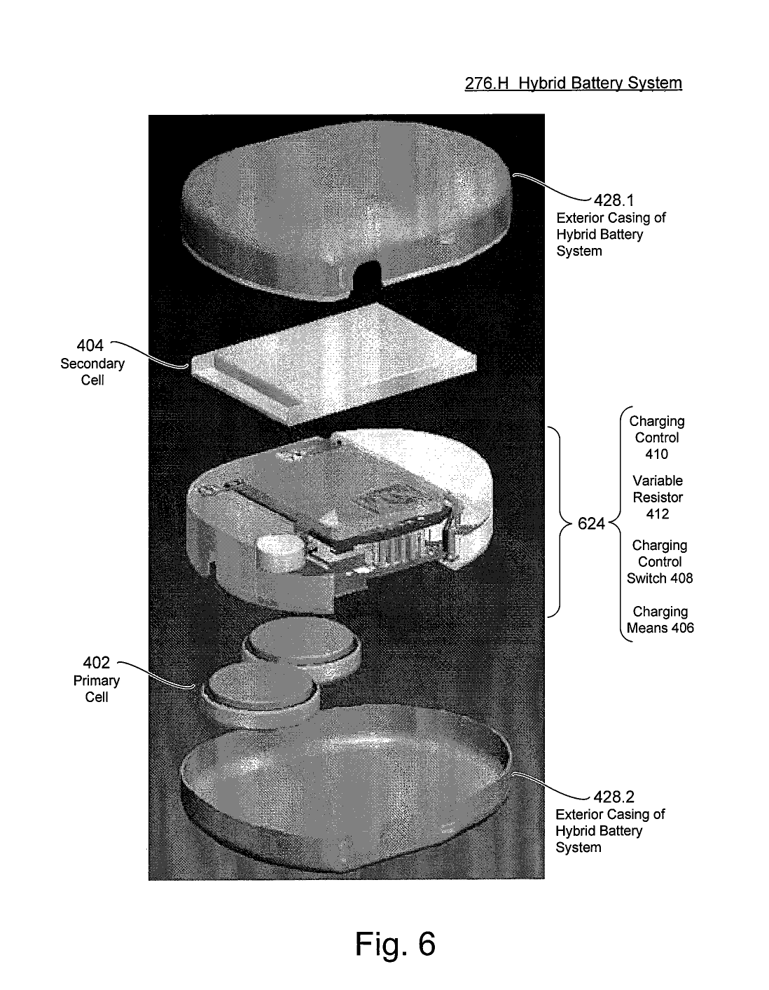

FIG. 6 presents an exploded view of an exemplary hybrid battery system 276.H according to an embodiment of the present system and method. As can be seen from the figure, hybrid battery system 276.H may include an exterior casing 428 which may include a first part 428.1 and a second part 428.2. First casing part 428.1 and a second casing part 428.2 may be configured to be coupled to each other, and to enclose the other elements of hybrid battery system 276.H, when hybrid battery system 276.H is fully assembled. Exterior casing 428 may also have openings for ports (not labeled) for power and data couplings.

Hybrid battery system 276.H may also include a primary cell 402, or in an alternative embodiment a plurality of primary cells 402. For example, shown in the figure are two primary cells 402. Having more than one primary cell provides additional storage capacity for longer life. Hybrid battery system 276.H may also include a secondary cell 404, or in an alternative embodiment a plurality of secondary cells 404.

In an embodiment, secondary cell 404 may be a lithium ion polymer cell. A lithium ion polymer cell uses an internal gel as an electrolyte, and may therefore be flat or configured in other shapes which lend themselves to a compact configuration for hybrid battery system 276.H. This is an advantage of the lithium ion polymer cell compared to other types of cells. For example, a standard lithium ion cell uses a liquid electrolyte, and so cannot readily be configured in a flat shape or other compact shapes.

Finally, hybrid battery system 276.H may include a circuit assembly 624. Circuit assembly 624 may include a number of elements already discussed above including, for example, and without limitation, charging means 406, charging control switch 408, secondary cell charging control circuit 410, variable resistor 412, and various buses and control lines already discussed above.

The present system and method pertains to a hybrid battery system which is substantially optimized for use with an ICTD 100. Exemplary embodiments of the present system and method have been described above in conjunction with FIGS. 4, 5, and 6.

### 7. Choice of Power Cells

Several elements distinguish the present system and method with respect to both prior batteries employed for use in ICTDs and to prior hybrid battery systems. Among these elements are the choices of power cells employed with the present system and method.

Persons skilled in the relevant arts will recognize that the term “battery” is sometimes employed in place of the word “cell” so that, for example, a “lithium ion polymer cell” may also be described, equivalently, as a “lithium ion polymer battery”. Within this document, individual batteries (lithium ion polymer, lithium/silver vanadium oxide, lithium magnesium oxide, etc.) are generally referred to as “cells” rather than batteries. This usage is strictly to help distinguish these cells from the overall hybrid battery system of the present system and method, which is comprised of multiple cells, and the usage (“cell” vs. “battery”) has no further significance.

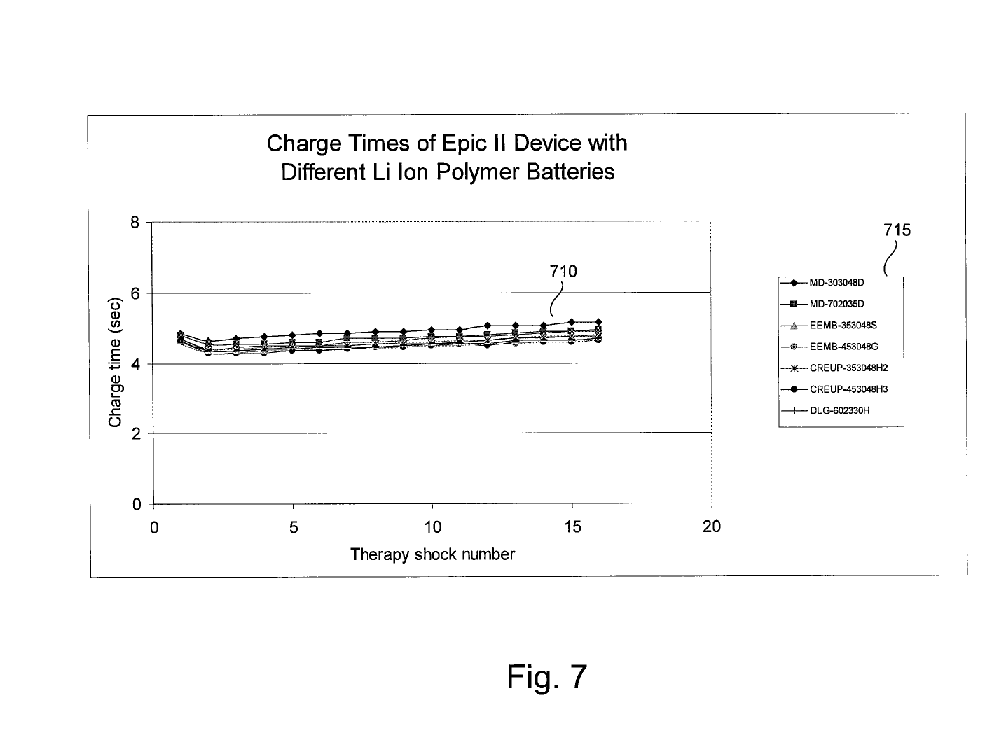

The inventors have investigated the performance properties of the Li ion polymer cell for use as the secondary cell in the context of charging shocking capacitors within an ICTD. FIG. 7 shows a set of plots 710 of the measured time required, in seconds, for various Li ion polymer cells (listed in legend 715 at right) to charge the shocking capacitors to approximately 750 to 800 volts in a representative ICTD (the Epic II ICD, manufactured by St. Jude Medical, Inc., of St. Paul, Minn.). The discharge current of the Li ion polymer cells was set at approximately 3 Amperes. As can be seen from plots 710, charging times were consistently at or below approximately 5 seconds, with only a slight increase in charging times over a series of shocks.

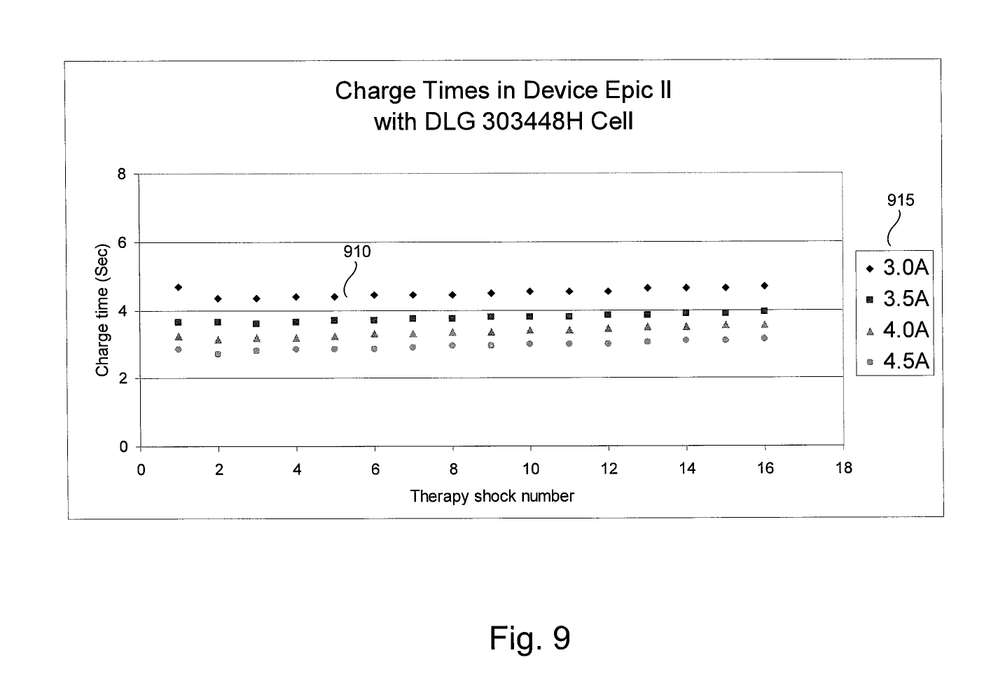

As discussed further below in conjunction with FIG. 9, charging times of approximately 5 seconds were specifically associated with a discharge current of approximately 3 Amperes. Emerging Li ion polymer cells are capable of significantly higher currents, of approximately 4 to 4.5 Amperes, which may result in charging times of approximately 2.5 to 3 seconds, or even less.

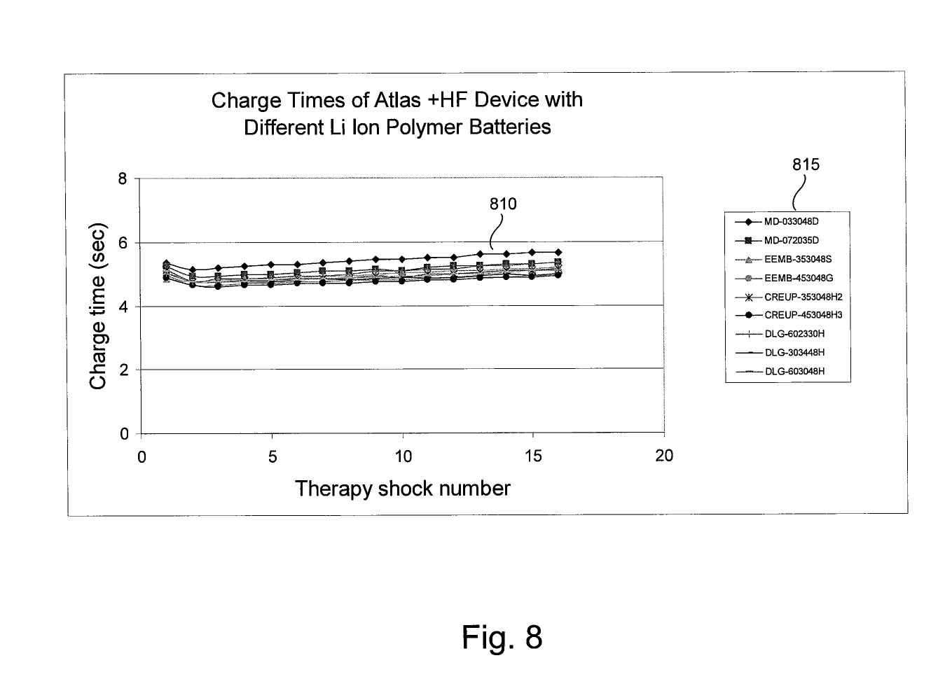

FIG. 8 shows a set of plots 810 of the time required, in seconds, for various Li ion polymer cells (listed in legend 815 at right) to charge the shocking capacitors to approximately 750 to 800 volts in another representative ICTD (the Atlas +HF ICD, manufactured by St. Jude Medical, Inc., of St. Paul, Minn.). Again, a current of approximately 3 Amperes from the Li ion polymer cells was employed. As can be seen from plots 810, charging times were consistently in the neighborhood of 5 seconds, and in many cases below 5 seconds with some of the cells tested.

FIG. 9 shows a set of plots 910 of the time required, in seconds, for a representative Li ion polymer cell (the DLG 303448H, manufacturer DLG Battery (Shanghai) Co., Ltd., Fengxian District, Shanghai, China) to charge the shocking capacitors to approximately 750 to 800 volts in a representative ICTD (the Epic II ICD, manufactured by St. Jude Medical, Inc., of St. Paul, Minn.). Different current levels (listed in legend 915) were employed, ranging from 3 Amps to 4.5 Amps. As can be seen from plots 910, charging times of well under 5 seconds could be achieved, in some cases being lower than 2.5 seconds.

As discussed in further detail below, a charge time of 5 seconds or less represents a significant improvement over charge times available with present systems using Li/SVO cells. As also discussed in further detail below, the Li ion polymer cell can provide current levels on the order of several Amps (for example, 3 to 5 Amps), thereby enabling the charge times on the order of 5 seconds or less, in some cases even less than 3.5 seconds, or even less than 3 seconds. Using a standard Li ion cell, current levels of 3 to 5 Amps could only be provided by a standard cell of undesirable size and weight, or a combination of multiple standard Li ion cells of undesirable size and weight, for the present application. Therefore, and as also discussed in further detail below, a Li ion polymer cell is to be preferred over a standard Li ion cell for the present system and method.

### 8. Lithium Ion Polymer Cell vs. Lithium/Silver Vanadium Oxide Cell

As already noted, the lithium/silver vanadium oxide (Li/SVO) cell has been used as a power source of ICTDs 100 for many years. While it has some desirable electrical properties, the internal resistance for both the anode and cathode increase as a result of the discharging process, particularly during midlife. This may ultimately result in premature battery replacement.

The Lithium ion polymer (Li ion polymer) cell, already described above as being used as the secondary cell 404 in exemplary embodiments of the present system and method, has both a higher voltage and lower internal resistance compared to the Li/SVO cell, making it desirable for use as the cell which charges shocking capacitor(s) 424 of ICTD 100.

In particular, the Li ion polymer cell has a higher current output than the Li/SVO cell. The discharge current of a typical Li/SVO battery used in an ICTD is approximately 3 Amps. A Li ion polymer battery may be discharged with a higher current, such as 3.5 to 4.5 Amps. Therefore, using the Li ion polymer cell as the power source 404 for the shocking capacitors 424, the discharge time, or equivalently, the time to charge the shocking capacitors 424, may be less than with the Li/SVO cell. For example, while it typically requires 10 to 18 seconds for a Li/SVO cell to charge the shocking capacitors 424 to approximately 750 volts to 800 volts, a Li ion polymer cell may charge the shocking capacitors to the same voltage (approximately 750 volts to 800 volts) in approximately 5 seconds, or even less time.

As described above, the present system and method employs a hybrid battery system 276.H utilizing two different types of power cells, a primary cell 402 and a secondary cell 404, in one package. In an embodiment, a secondary cell 404 which may be a Li ion polymer cell is continuously charged by one or more physically small primary battery cells 402, which may be Lithium Magnesium Oxide (Li/MnO₂) cells or Lithium Carbon Monoflouride (LiCFₓ) cells. The discussion below generally refers to the Li/MnO₂ cell as the primary cell, it being understood that in some embodiments of the present system and method, the LiCFₓ or other cells may be employed instead as primary cell 402.

Charging means 406 is employed to charge the secondary cell from the primary cell. In an embodiment, the charging means 406 is a DC-to-DC converter 406, and the secondary cell 404 is charged by the primary cell 402 via DC-to-DC converter 406. DC-to-DC converter 406 steps up the voltage going from the primary cell 402 to the secondary cell 404.

A Li ion polymer battery with, for example, LiCoO₂ cathode material, may be recharged up to 4.23V. This is about one volt higher than a new Li/SVO battery. The internal resistance of a Li ion polymer battery may be lower than 0.1 Ω. In an embodiment, the output voltage of DC-to-DC converter 406 is set at approximately 4.2 volts. In this way, Li ion polymer cell 404 can be maintained at an unloaded voltage higher than 4.0 volts. A further advantage of the Li ion polymer cell is that, unlike with the Li/SVO cell, there is no significant increase in internal resistance over the life of the Li ion polymer cell. Therefore, in the capacitor charging process the voltage drop will be less, and the loaded voltage remains higher over the life of the Li ion polymer cell, as compared with the Li/SVO cell. The unloaded voltage on the Li ion polymer cell can be maintained at approximately 4.1 to 4.2 volts, while the loaded voltage, during charging of shocking capacitor(s) 424, may be maintained at approximately 3.5 volts.

As a result of all these combined advantages of the Li ion polymer cell, the discharge time for high voltage shocking (that is, the time to charge shocking capacitor(s) 424) will be significantly less compared to the discharge time using a Li/SVO battery. The time to charge the shocking capacitors is approximately 10 to 20 seconds for the Li/SVO cells presently in use. Charging times of approximately 5 seconds or even less, such as less than 4 seconds or less than 3.5 seconds, may be achieved with the Li ion polymer cell.

As described above, secondary cell 404 is used to charge the shocking capacitor(s) 424 when cardiac shocking is required. The primary cell 402, in addition to continuously charging the secondary cell, is also used to power background operations of ICTD 100. Such background operations may include cardiac monitoring, cardiac pacing, and various communications, data processing, and other maintenance activities of the ICTD 100.

In an embodiment of the present system and method, any control processing related to cardiac shocking may be powered by secondary cell 404. In an alternative embodiment, control processing related to cardiac shocking may be powered in part or in whole by primary cell 402, but charging of shocking capacitor(s) 424 is still performed by secondary cell 404.

In summary, compared to the Li/SVO battery which has been used to charge shocking capacitor(s) 424 in the past, the Lithium ion polymer cell has the following advantages as the secondary cell 404: (i) higher loaded voltage compared to the Li/SVO battery; (ii) lower internal resistance compared to Li/SVO battery; (iii) higher discharge current during capacitor charging; (iv) reduced discharge time during capacitor charging; (v) faster voltage recovery (faster charging time); and (vi) lower cost.

### 9. Lithium Ion Polymer Cell vs. Standard Lithium Ion Cell

Li ion polymer cells also offer advantages as a secondary cell 404, as compared with standard Li ion cells that might be considered for use in the same capacity (that is, as a candidate for secondary cell 404).

Because Li ion polymer cells use gelatinous electrolyte, their self-discharge rate is relatively lower than that of a regular Li ion battery. (The self-discharge rate reflects the rate at which a cell spontaneously loses power, even with no external load or usage, due to internal chemical reactions.) The self-discharge rate of the Li ion polymer cell is in the range from 2% to 5% per month. The self-discharge rate of the standard Li ion cell is in the range of 5% to 10% per month. Because the Li ion polymer cell has a lower self-discharge rate, it will require less electrical charge from primary cell 402 (as compared with the charge that would be required if the standard Li ion cell were employed as secondary cell 404). Since less charge is required from primary cell 402, more power is preserved in primary cell 402. This enhances the overall functional lifetime of hybrid battery system 276.H.

Also, and as noted above, Li ion polymer cells can be manufactured in thin, pliable shapes that offer advantages in device packaging compared with standard Li ion batteries, which have more bulk and are generally of rigid construction.

For typical shocking purposes, a desired storage of a secondary cell might be 250 milliampere-hours. This is more than sufficient to provide power for a series of six shocks during a defibrillation process. A standard lithium ion cell might have a discharge current capacity of 1 C to 2 C, meaning that it can only provide current at a rate equivalent to its storage capacity, or at most twice its storage capacity. For example, a standard Li ion cell with a storage capacity of 250 milliampere-hours and a discharge current of 2 C can provide at most 500 milliamps of current. At such a current flow, it may take a minute or several minutes to charge the shocking capacitors. This is insufficient for real-world applications, so a larger cell (or additional cells) would be required.

By contrast, an exemplary Li ion polymer cell may have a discharge current capacity of anywhere from 5 C to 20 C, or even higher. At this discharge current capacity, the Li ion polymer cell may be able to discharge at a rate from 5 times to 20 times its storage capacity. Again assuming a total cell power storage of 250 milliampere-hours, an exemplary Li ion polymer cell can deliver a current from 1.25 amps (for a 5 C cell) to 5 amps (for a 20 C cell). It may be possible to achieve a shocking capacitor charge time of as short as 5 seconds or even less, such as approximately 3.5 seconds, 3 seconds, or even less. This is a dramatic improvement over the charge times of approximately 10 to 20 seconds achieved with presently used Li/SVO batteries. As shown in FIG. 9 (already discussed above), with some Li ion polymer cells it may be possible to charge shocking capacitor(s) 424 in times under 3 seconds, and possibly even under 2.5 seconds, which is much less than the charge times available with present devices.

### 10. Charging of Lithium Ion Polymer Cell from Primary Cell

In an embodiment, one or more primary Li/MnO2 button cell(s) 402 is (are) connected with a Li ion polymer cell 404 in parallel through a DC-to-DC converter 406 (see FIGS. 4 and 5). Both cells, along with the DC-to-DC converter, are packaged in one device 276.H comprising the hybrid battery system 276.H. The entire system is enclosed in exterior casing 428. (See FIGS. 4, 5, and 6.) Except during the brief time periods when cardiac shocking may be in progress, the Li ion polymer cell 404 is continuously charged by the small Li/MnO2 cell(s) 402 with a low current flow, typically at milliampere levels.

An advantage of the present system and method is that the Li ion polymer cell 404 can be continuously charged, meaning that little or no additional circuitry is required to regulate the rate of charging. A secondary cell charging control circuit 410 may be present to ensure that the Li ion polymer cell 404 is disconnected from primary cell 402 when cardiac shocking is in progress. However, in normal usage of an ICTD 100, cardiac shocking is not in progress the great majority of the time.

By default, primary cell 402 is coupled to secondary cell 404, and during those intervals when cardiac shocking is not in progress, secondary cell charging control circuit 410 is configured to automatically enable the default coupling between the primary cell 402 and the secondary Li ion polymer cell 404. This ensures that the secondary cell 404 is charged by the primary cell 402. Variable resistor 412 may limit the current flow from primary cell 402 to secondary cell 404. When using a Li ion polymer cell as the secondary cell 404, no other control, regulation, or rate monitoring of the charging process is required to ensure the safe charging of secondary cell 404. This greatly simplifies the design of hybrid battery 276.H in terms of both design complexity and cost.

### 11. Relative Storage Capacities of Different Types of Cells

In an embodiment of the present system and method, the size and capacity of the two different types of cells (primary and secondary) are appropriately selected.

Over the life of a typical ICTD 100, about 25% to 30% of total ICTD battery capacity is used for high voltage shocking; the other 70% to 75% of capacity is used for pacing and background operation and reforming the electrolytic capacitors. It is desirable, over the life of the ICTD, to maintain the available voltages from the hybrid battery system, at suitably high respective levels for both background operation and cardiac shocking.

Regarding the secondary cell 404, it is an advantage of the present system and method to select the size of the Li ion polymer cell such that the cell provides approximately 25-30% of the total initial power storage capacity of the hybrid battery, for example, around 400 milliampere-hours. Other capacity Li ion batteries may be used, ranging from about 150 milliampere-hours up to 600 milliampere-hours, depending upon the desired tradeoffs in device volume vs. charge time, and the total number of sequential high voltage charge capabilities needed.

In general, however, it is desirable to avoid a Li ion polymer battery with too small a capacity. If the size is too small, the internal resistance will be higher, and that will negatively impact the discharge rate. In addition, if a patient requires a large number of shocks in a short time, a secondary cell 404 which is too small will be unable to provide the required number of shocks.

The primary cell is selected to provide approximately 70% to 75% of the total initial power storage of the hybrid battery system. The use of the Li/MnO₂ cell as primary cell 402 also has advantages. Its voltage is high, with a nominal voltage of about 3.0 volts. The energy density is also relatively high. It has long storage life. Its cost is low. A Li/MnO₂ button cell with 1000 milliampere-hours or two button cells with 550 milliampere-hours each are selected to charge the Li ion polymer battery. The Li/MnO₂ button cells can be discharged with current at milliampere level, which is appropriate for charging purpose. Other capacity primary cells may be used to obtain the desired device longevity, depending upon expected usage and average current consumption by the device.

### 12. Alternative Embodiments

In an embodiment of the present system and method, each primary cell 402 (for example, lithium magnesium oxide cell(s), etc.) and each secondary cell 404 (for example, lithium ion polymer cell(s)) is a self-contained, sealed battery unit, of a kind which may be purchased off-the-shelf and readily coupled to conventional electrical contacts in a larger system. In an alternative embodiment, either or both of the primary cell or the secondary cell may be specially constructed from custom parts or elements, specifically tailored for integration into the hybrid battery system of the present system and method. The details of such construction, if any, are beyond the scope of this document.

In embodiments described above, the hybrid battery system employs a single type of primary cell 402 for powering background operations and for charging secondary cell 404, and also employs a single type of secondary cell 404 for charging shocking capacitor(s) 424. In an alternative embodiment, more than one type of primary cell may be employed for powering different types of background operation circuitry 430 or for charging different types of secondary cells 404.

In an alternative embodiment, a first type of primary cell 402 may be employed to provide power to background operation circuitry 430, and a second type of primary cell 402 may be employed to charge secondary cell 404.

In an alternative embodiment, different types of secondary cells 404 may be employed, which may be suitable for different types, patterns, time durations, or required power levels of shocking activity. Suitable switching and/or coupling circuitry may be employed to select and support the additional types of power cells as appropriate.

### 13. Conclusion

It is to be appreciated that the Detailed Description section, and not the Summary and Abstract sections, is intended to be used to interpret the claims. The Summary and Abstract sections may set forth one or more but not all exemplary embodiments of the present system and method as contemplated by the inventor(s), and thus, are not intended to limit the present method and system and the appended claims in any way.

Moreover, while various embodiments of the present system and method have been described above, it should be understood that they have been presented by way of example, and not limitation. It will be apparent to persons skilled in the relevant art(s) that various changes in form and detail can be made therein without departing from the spirit and scope of the present system and method. Thus, the present system and method should not be limited by any of the above described exemplary embodiments, but should be defined only in accordance with the following claims and their equivalents.

In addition, it should be understood that the figures and screen shots illustrated in the attachments, which highlight the functionality and advantages of the present system and method, are presented for example purposes only. The architecture of the present system and method is sufficiently flexible and configurable, such that it may be utilized (and navigated) in ways other than that shown in the accompanying figures. Moreover, the steps indicated in the exemplary system(s) and method(s) described above may in some cases be performed in a different order than the order described, and some steps may be added, modified, or removed, without departing from the spirit and scope of the present system and method.

Further, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the present system and method in any way.

What is claimed is:

1. A hybrid system battery configured to power an implantable cardiac therapy device (ICTD), comprising:
a primary cell;
a rechargeable secondary cell coupled to the primary cell; and
charging means configured to charge the secondary cell from the primary cell, wherein the primary cell is configured to power background operation circuitry of the ICTD; and
wherein the secondary cell is configured to provide power for high voltage shocking.

2. The hybrid battery system of claim 1, wherein the secondary cell is configured to provide power to at least one of a shocking circuit of the ICTD or a shocking capacitor of the ICTD.

3. The hybrid battery system of claim 1, wherein the secondary cell is configured to be charged via an unregulated charging process.

4. The hybrid battery system of claim 1, wherein the secondary cell is configured to be charged via a continuous charging process.

5. The hybrid battery system of claim 1, wherein the charging means comprises a direct-current-to-direct-current (DC-to-DC) converter.

6. The hybrid battery system of claim 1, wherein the secondary cell is configured to provide at least one of a higher voltage or a higher current than the primary cell.

7. The hybrid battery system of claim 1, wherein the secondary cell is configured to charge a shocking capacitor of the ICTD to a desired voltage in a time less than approximately 5 seconds.

8. The hybrid battery system of claim 1, wherein the secondary cell is further configured to charge a shocking capacitor of the ICTD to a desired voltage in a time less than approximately 3.5 seconds.

9. The hybrid battery system of claim 1, wherein the secondary cell is configured to deliver to a shocking circuit of the ICTD at least one of a current of at least approximately 4 amperes or a loaded voltage of at least approximately 3.5 volts.

10. The hybrid battery system of claim 1, wherein the secondary cell comprises a Lithium ion polymer cell.

11. The hybrid battery system of claim 10, wherein the primary cell comprises at least one of a Lithium-Magnesium Oxide (Li/MnO2) cell or a Lithium Carbon Monoflouride (LiCFx) cell.

12. The hybrid battery system of claim 1, wherein the primary cell is configured to initially store approximately 70% to 75% of a total initial energy storage of the hybrid battery system, and the secondary cell is configured to initially store approximately 25% to 30% of a total initial energy storage of the hybrid battery system.

13. The hybrid battery system of claim 1, wherein the charging means maintains the secondary cell at a voltage greater than a voltage of the primary cell, wherein the unloaded voltage of the secondary cell is maintained at a voltage of at least 4 volts.

14. The hybrid battery system of claim 1, further comprising a charging control circuit, wherein:
the charging control circuit is configured to automatically decouple the secondary cell from the primary cell when the secondary cell is delivering a current for shocking, wherein only the secondary cell delivers a current to a shocking circuit of the ICTD during a defibrillation process; and
the charging control circuit is configured to automatically recouple the secondary cell to the primary cell when the secondary cell has finished delivering the current for shocking.

15. The hybrid battery system of claim 14, wherein the secondary cell is configured to be continuously coupled to the primary cell when the secondary cell is not delivering the current for shocking.

16. The hybrid battery system of claim 1, wherein:
the background operation circuitry comprises at least one of a monitoring circuitry of the ICTD or a pacing circuitry of the ICTD; and
the primary cell is configured to directly power at least one of the monitoring circuitry or the pacing circuitry.

17. The hybrid battery system of claim 1, wherein the secondary cell is configured to provide power to a high voltage charging circuit of the ICTD, the high voltage charging circuit being configured to step up the voltage from the secondary cell to a voltage suitable for cardiac shocking.

18. The hybrid battery system of claim 1, wherein:
the secondary cell is configured to provide power to a shocking circuit of the ICTD; and
the shocking circuit comprises:
a high voltage capacitor configured for cardiac shocking; and
a high voltage charging circuit configured to charge the high voltage capacitor to a voltage suitable for cardiac shocking.

19. The hybrid battery system of claim 1, wherein the primary cell is further configured to power a control circuitry which regulates a shocking process which is powered by the secondary cell.

20. An implantable cardiac therapy device (ICTD) comprising:
a shocking circuit;
a background operation circuit;
a primary cell configured to provide power to the background operation circuit;
a rechargeable secondary cell configured to provide power to the shocking circuit for high voltage shocking; and
a power converter configured to charge the secondary cell from the primary cell.

21. The ICTD of claim 20, wherein the secondary cell comprises a Lithium ion polymer cell.

22. The ICTD of claim 20, wherein the primary cell comprises at least one of a Lithium-Magnesium Oxide (Li/MnO2) cell or a Lithium Carbon Monoflouride (LiCFx) cell.

23. The ICTD of claim 20, wherein the primary cell is configured to initially store approximately 70% to 75% of a total initial energy storage of the ICTD, and the secondary cell is configured to initially store approximately 25% to 30% of the total initial energy storage of the ICTD.

24. The ICTD of claim 20, wherein the shocking circuit comprises a high voltage capacitor configured for cardiac shocking, and a high voltage charging circuit configured to charge the high voltage capacitor to a voltage suitable for cardiac shocking.

25. The ICTD of claim 20, wherein:
the shocking circuit comprises a shocking capacitor and a control circuit configured to regulate a shocking process;
the secondary cell is configured to provide power to the shocking capacitor for the shocking process; and
the primary cell is configured to provide power to the control circuit to regulate the shocking process.

26. A method for powering an implantable cardiac therapy device (ICTD), comprising:
delivering power to background operation circuitry of the ICTD from a primary cell;
delivering power to a shocking capacitor of the ICTD from a secondary cell; and
charging the secondary cell from the primary cell.

27. The method of claim 26, wherein the second power delivering step comprises delivering power from a Lithium ion polymer cell.

28. The method of claim 26, wherein the first power delivering step comprises delivering power from a Lithium-Magnesium Oxide (Li/MnO2) cell or a Lithium Carbon Monoflouride (LiCFx) cell.

29. The method of claim 26, wherein the step of delivering power to the shocking capacitor comprises charging the capacitor to a desired voltage in a time less than approximately 5 seconds.

30. The method of claim 26, wherein the step of delivering power to the shocking capacitor of the ICTD from the secondary cell comprises:
delivering power from the secondary cell to a high voltage charging circuit; and
at the high voltage charging circuit, stepping up a voltage delivered from the secondary cell to a voltage suitable for cardiac shocking.
